(19) United States
(12) Patent Application Publication
(10) Pub. No.: US 2020/0367845 A1
(43) Pub. Date: Nov. 26, 2020

(54) PROSTATE IMAGING SYSTEM AND METHOD

(71) Applicants: Alex DOLGONOS, Thornhill (CA); Karen SAFARYAN, Concord (CA); Aaron FENSTER, London (CA)

(72) Inventors: Alex DOLGONOS, Thornhill (CA); Karen SAFARYAN, Concord (CA); Aaron FENSTER, London (CA)

(21) Appl. No.: 16/484,381

(22) PCT Filed: Feb. 7, 2018
(86) PCT No.: PCT/CA2018/050138

§ 371 Date: Oct. 4, 2019

Related U.S. Application Data
(60) Provisional application No. 62/455,665, filed on Feb. 7, 2017.

(51) Int. Cl. A61B 6/00; A61B 6/03; A61B 6/06; A61B 6/04; G01T 1/24; G01S 15/89

(52) U.S. Cl. CPC A61B 6/50; A61B 6/037; A61B 6/06; G01S 15/8993; A61B 6/0407; A61B 6/5247; G01T 1/24; A61B 6/0478

(57) ABSTRACT

According to an example aspect is an imaging system for tomographic imaging of a body portion of a patient after administration of a radiopharmaceutical, the system comprising: a surface for supporting the body portion of the patient. The system further comprises a detector assembly positioned proximate the body portion. The detector assembly comprises at least one solid-state detecting unit, which itself comprises a nuclear detector and a collimator defining a solid collection angle. The system further comprises a processor for receiving collected data from the detector assembly.

### FIELD

This description relates to a system and method of nuclear imaging of the prostate region, optionally combined with structural imaging.

### BACKGROUND OF THE INVENTION

Prostate cancer is one of the most prevalent cancers to affect the male population. A blood test measuring the level of prostate specific antigen (PSA) is widely used for screening for the disease in males. However, since the PSA test is tissue-based and not cancer-based, the benefits of PSA tests are controversial as they have a high false-positive rate, often resulting in over-diagnosis and unnecessary treatment.

Other diagnostic procedures typically used along with the PSA test include a digital rectal exam, a transrectal ultrasound and/or imaging-guided biopsy. However, these procedures often put the patient in a great deal of discomfort, since both the digital rectal exam and the transrectal ultrasound require an implement to be inserted into the rectum as part of the procedure. Transperineal biopsies, in particular, are also painful to undergo. Yet due to the nature of prostate cancer in which the prostate gland may contain multiple tumours, the random sampling procedure has a high probability of missing small cancerous tissue bodies in the prostate.

Methods of nuclear medical imaging can be used to more accurately image the structure and functionality of organs, including computed tomography CT, magnetic resonance imaging (MRI), positron emission tomography (PET), and single photon-emission computed tomography (SPECT). However, their applications in imaging the prostate region, and subsequent diagnosing of prostate cancer, is limited due to the small size of the prostate gland and the large distance between the organ and the detectors used in these imaging systems. During the imaging process, the scintillation detectors are often positioned 50cm or more from the imaged organ. At such distances, the detector's effectiveness at detecting radiation is low, resulting in low spatial resolution. Moreover, these systems are often very expensive and cumbersome to use.

One known possible solution are Cadmium Zinc Telluride (CdZnTe or CZT) radiation detectors. CZT has several advantages in nuclear imaging. CZT is a semiconductor material with wide band-gap and relatively high electron mobility. CZT material also has high stopping power for gamma rays. In use for medical imaging, therefore, a CZT material of only few millimeters often affords sufficient detection efficiency.

Known uses of CZT thus far, however, involve the integration of CZT into trans-rectal probes. While this allows imaging of the prostate gland at a short distance of 1-5 cm, it still requires trans-rectal insertion of an implement, which tends to cause discomfort in patients.

### SUMMARY

According to an example aspect is an imaging system for tomographic imaging of a body portion of a patient after administration of a radiopharmaceutical, the system comprising: a surface for supporting the body portion of the patient. The system further comprises a detector assembly positioned proximate the body portion. The detector assembly comprises at least one solid-state detecting unit, which itself comprises a nuclear detector and a collimator defining a solid collection angle. The system further comprises a processor for receiving collected data from the detector assembly.

According to another example is a method for non-invasive imaging of a body portion of a patient after administration of a radiopharmaceutical agent into the patient. The method comprises aligning the body portion of the patient adjacent a detector assembly and imaging the body region of the patient using at least one solid-state detecting unit. The solid-state detecting unit comprises a nuclear detector and a collimator defining a solid collection angle. The method further comprises processing the collected image data from the detector assembly.

### BRIEF DESCRIPTION OF THE DRAWINGS

Example embodiments of the invention are provided in the following description. Such description makes reference to the annexed drawings wherein:

FIG. 1 is a bottom-front perspective view of an example embodiment of a system for imaging of a body portion of a patient according to the present invention.

FIG. 2 is a top-front perspective view of the system of FIG. 1.

FIG. 3 is a bottom view of the system of FIG. 1.

FIG. 4 is a top view of the system of FIG. 1.

FIG. 5 is a side view of the system of FIG. 1.

FIG. 6 is a back-side view of the system of FIG. 1.

FIG. 7 is an enlarged view of area A of FIG. 5.

FIG. 8 is a side plan view of another example embodiment of a system in use for imaging of a body portion of a patient according to the present invention.

FIG. 9 is an enlarged view of a portion of FIG. 8.

FIG. 10 is a top view of the system of FIG. 8 in use.

FIG. 11 is a perspective view of a solid-state detector unit as used in an example embodiment of the present invention.

FIG. 12 is an end plan view of the solid-state detector unit of FIG. 11.

FIG. 13 is a side plan view of the solid-state detector unit of FIG. 11.

FIG. 14 is an enlarged view of area B of FIG. 13.

FIG. 15 is a perspective view of a collimator as used in an example embodiment of the present invention.

FIG. 16 is an end plan view of the collimator of FIG. 15.

FIG. 17 is a side plan view of the collimator of FIG. 15.

FIG. 18 is a cross-sectional view of FIG. 17 along line C-C.

FIG. 19 is a schematic view of an embodiment of the imaging system according to the present invention.

FIG. 20 is a flow chart of a method according to the present invention.

FIG. 21 is an example 3D ultrasound image of a prostate taken using a system of the present invention.

FIG. 22 is an example 3D image of radionuclide labels tracer registered to the 3D ultrasound image of FIG. 21 taken using a system of the present invention.

FIG. 23 is the 3D ultrasound image of the prostate of FIG. 21 with the 3D image of the tracer of FIG. 22 superimposed.

### DESCRIPTION OF EXAMPLE EMBODIMENTS

The Figures illustrate exemplary embodiments of an imaging system for tomographic imaging of a body portion of a patient after administration of a radiopharmaceutical according to the present invention. The structures of the systems will first be described, and then a method of their use will be discussed.

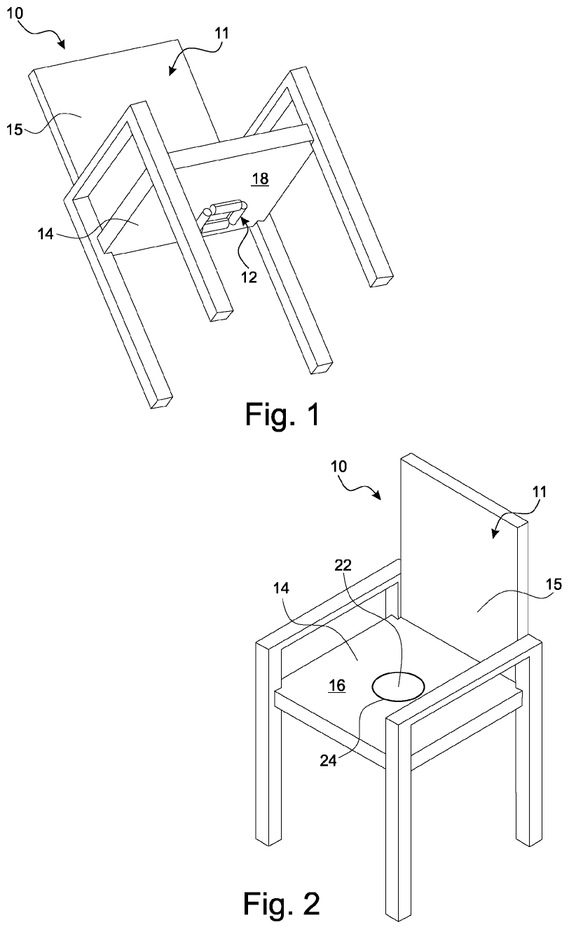

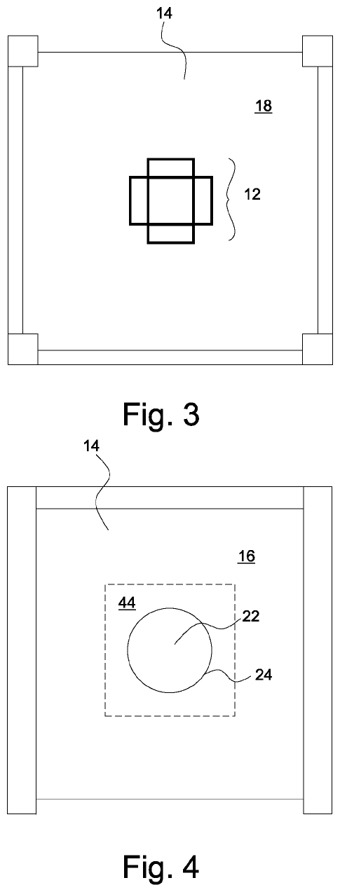

The imaging system of the present invention mainly includes a surface 9 for supporting a body portion of a patient, a detector assembly 12 positioned proximate the body portion and a processor 13 for receiving collected data from detector assembly 12. As seen in FIGS. 1 to 6 an imaging system 10 of an example embodiment of the system of the present invention is shown. With imaging system 10 surface 9 is part of a chair 11.

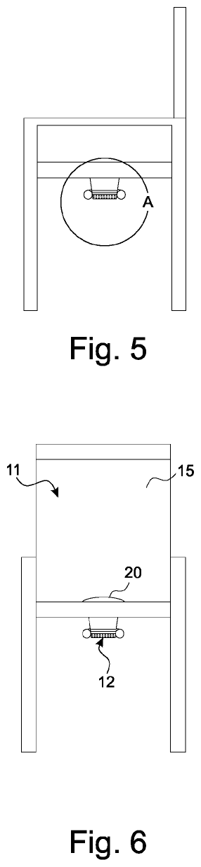

Chair 11 comprises a seat 14 for supporting a body portion of the patient and a back 15 fixed to an edge of seat 14. In the particular embodiment shown, seat 14 supports a buttocks region of the patient (not shown) when the patient is sitting in chair 11. Seat 14 has a support surface 16 and an opposed surface 18. In order to better conform to the shape of the buttocks region of the patient and provide greater surface area contact between the support surface and the buttocks of the patient, support surface 16 has a raised central ridge 20 (see FIGS. 5 and 6) generally bisecting seat 14. Seat 14 may optionally also have curved depressions flanking central ridge 20 (not shown) to provide even greater surface area contact between the support surface and the buttocks of the patient. Raised central ridge 20 may extend around both sides of opening 22. In some example's the ridge 20 and/or other contours of the seat may be adjustable to accommodate different patient body types.

Seat 14 further defines an opening 22 having a periphery 24. As shown in FIGS. 2 and 4, opening 22 in the depicted embodiment is positioned generally in the centre of seat 14. In an alternative exemplary embodiment, not shown, opening 22 could be positioned in seat 14 towards back 15. In this way, opening 22 would be proximate a tailbone area of the sitting patient.

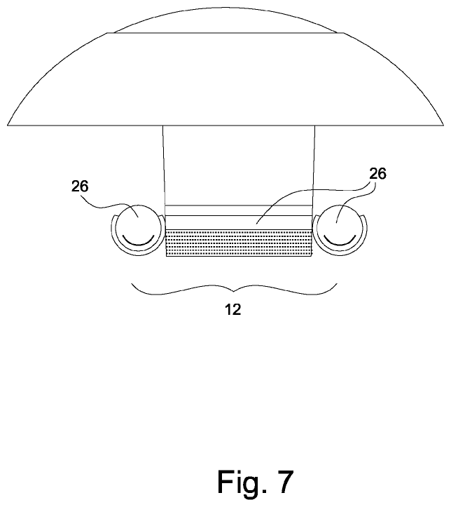

Detector assembly 12, as depicted in FIGS. 1, 3, and 7, comprises four solid-state detecting units 26. Solid-state detecting units 26 are arranged to follow periphery 24 of opening 22 and generally surround opening 22. Of course, one skilled in the art would recognize that any number of detecting units 26 in a different arrangement may be used in this manner so long as they are proximate opening 22, and importantly, proximate to the prostate region of the patient.

In this particular embodiment, detector assembly 12 is mounted to opposed surface 18 proximate periphery 24 of opening 22. However, it is not necessary for detector assembly 12 to be mounted to opposed surface 16. One skilled in the art would understand that so long as detector assembly 12 is proximate to opening 22 (i.e. proximate the prostate region), detector assembly 12 may be supported in such a position via any number of means, including detector assembly 12 being elevated from the ground or detector assembly 12 being fixed to an adjacent wall etc.

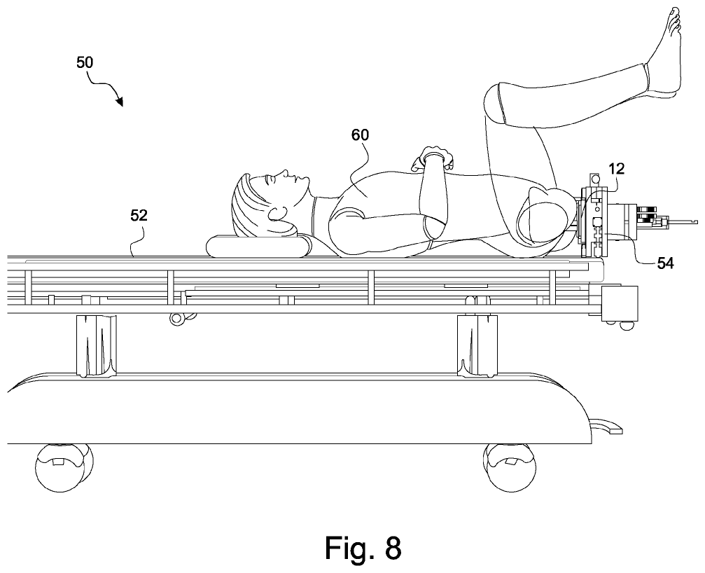

As an alternative to the chair configuration shown in FIGS. 1-7, FIGS. 8-10 show an imaging system 50 having a different configuration. In this depicted embodiment, imaging system 50 includes a surface for supporting the body portion of a patient 60, in this particular example, the prostate region. The surface is shown to be a component of a bed 52 on which the patient may be lying on his side, back or front. In FIG. 8, patient 60 is lying on his back with his legs extending outwardly, generally perpendicular from bed 52.

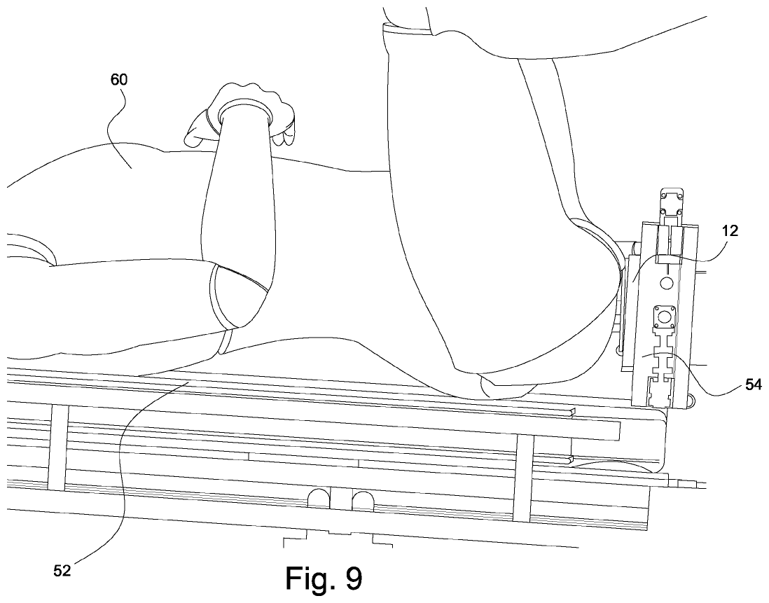

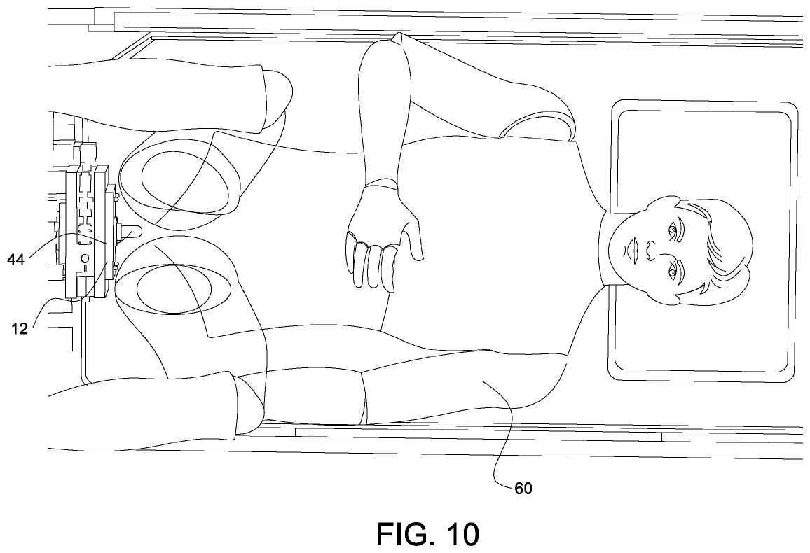

Similar to imaging system 10 discussed above, imaging system 50 includes detector assembly 12, which itself includes solid-state detecting units (not shown) positioned proximate the prostate region. As best seen in FIGS. 9 and 10, detector assembly 12 is orientated approximately perpendicular from bed 52 and is supported by an arm 54, which is secured to bed 52. The extension and spreading of the patient's legs outwardly and generally parallel to detector assembly 12, while lying on bed 52 allows detector assembly 12 to be brought into close proximity to the prostate region of patient 60.

Detector assembly 12 in this depicted embodiment is similar to the detector assembly described above in that it also includes one or more solid-state detecting units which each have a nuclear detector and a collimator defining a solid collection angle. Imaging system 50 also includes a processor (not shown) for receiving collected data from the detector assembly. In the shown embodiment, the solid-state detecting units are embedded within the structure of detector assembly 12.

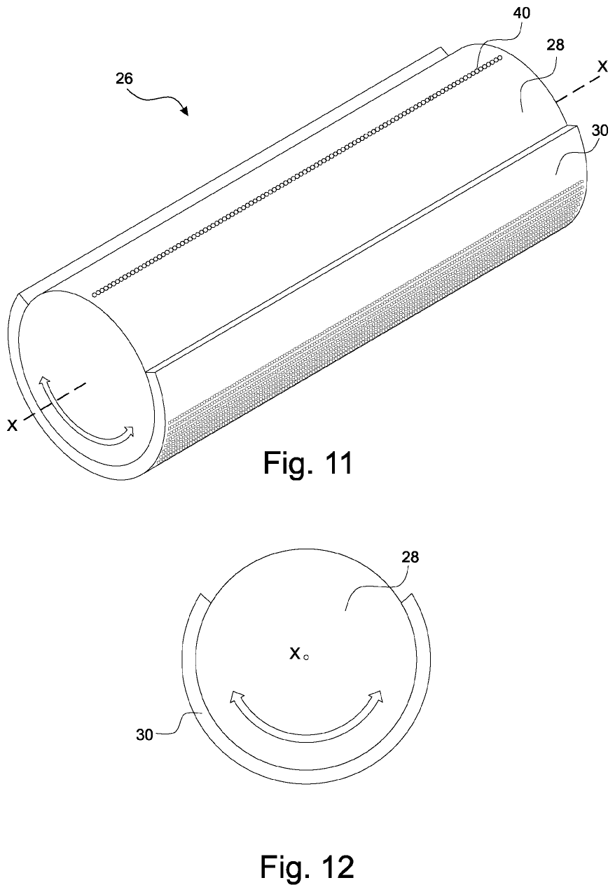

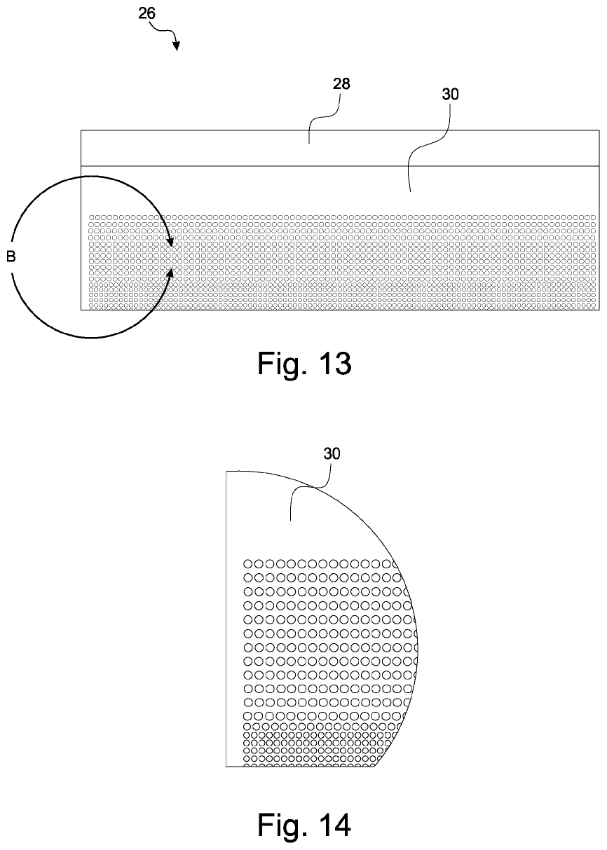

Turning now to the solid-state detecting units, as shown in FIGS. 11 to 14, each solid-state detecting unit 26 comprises a nuclear detector and a collimator 28 defining a solid collection angle. The nuclear detector in the embodiment shown is a cadmium zinc telluride (CZT) based radiation detector 30, also known as a gamma detector. CZT radiation detector 30 as shown is configured into a partial cylindrical shape so that it longitudinally “wraps” around collimator 28. Each solid-state detecting unit 26 of this embodiment may measure between approximately 3 to 4 inches long. Solid-state detecting unit 26 in the depicted embodiment is 3.15 inches long. By way of example, CZT radiation detector 30 may incorporate a CZT radiation detector available purchased from Redlen Technologies.

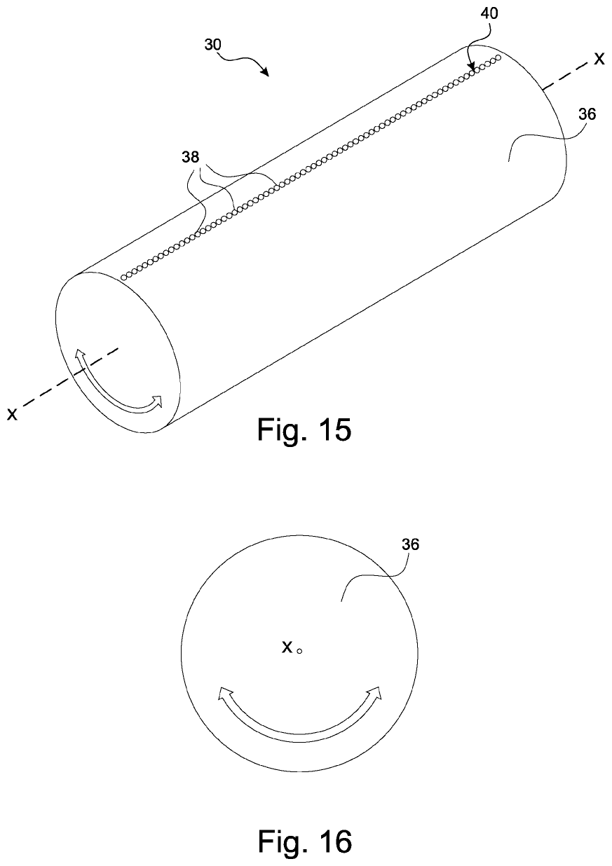

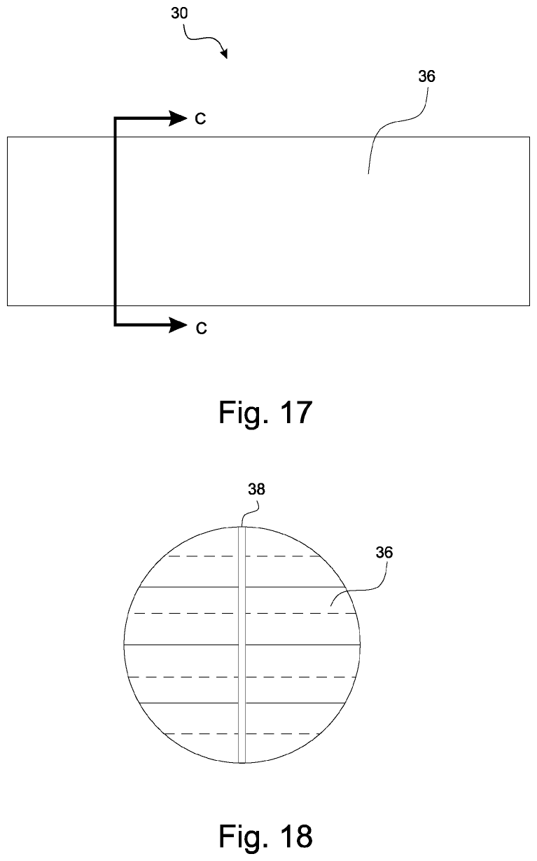

Collimator 28 of the present embodiment is depicted in isolation in FIGS. 15 to 18. As shown, collimator 28 comprises a generally cylindrical body 36 having a longitudinal axis X and a diameter of approximately between 0.3 to 0.4 inches. Collimator 28 of the depicted embodiment has a diameter of 0.393 inches. As indicated by the rotating arrow in FIGS. 11 and 12, collimator 28 can rotate around axis X relative to CZT radiation detector 30.

Cylindrical body 36 further defines multiple apertures 38 longitudinally disposed along the length of cylindrical body 36. Apertures 38 are arranged along cylindrical body 36 to form a line 40, which defines the solid collection angle. As best seen in FIG. 18, each aperture 38 extends diametrically across cylindrical body 36, positioned parallel to one another. In the depicted embodiment, each aperture 38 has a diameter of approximately 0.02 inches. As understood by the skilled person, while specific measurements are noted, radiation detectors and collimators of differing proportions and sizes may be used depending on their application.

Apertures 38 of line 40 collectively collimate, or will allow, photons traveling at the “right” or desired angle to pass through collimator 28 and strike CZT radiation detector 30, generating an image. Collimator 28 may be made of tungsten, lead, or any other material that absorbs photons/gamma rays not traveling at the desired angle.

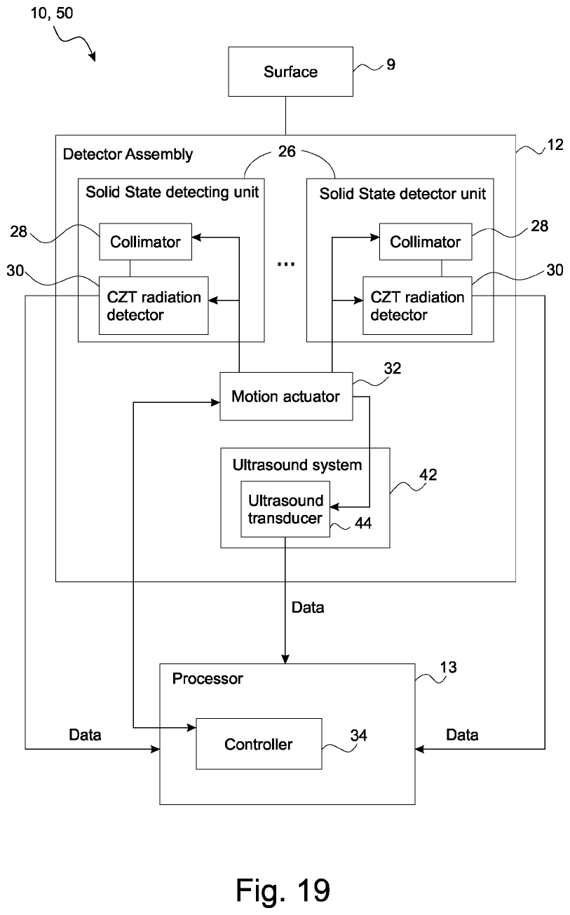

Imaging system 10/50 is shown schematically in FIG. 19, where detector assembly 12 may include one or more solid state detecting units 26 and a processor 13 is coupled to detector assembly 12 to receive the imaging data from CZT radiation detectors 30 of detector assembly 12.

Detector assembly 12 further comprises a motion actuator 32 coupled to solid-state detecting unit 26. Motion actuator 32 may be coupled to collimator 28 and configured to rotate collimator 28 about axis X relative to CZT radiation detector 30 during data acquisition. In another example, motion actuator 32 may be coupled to CZT radiation detector 30 and configured to rotate CZT radiation detector 30 about axis X relative to collimator 28 during data acquisition. In yet another example, motion actuator 32 may be coupled to both collimator 28 and CZT radiation detector 30 to allow collimator 28 and CZT radiation detector 30 to rotate about axis X relative to one another during data acquisition.

Alternatively, motion actuator 32 may be coupled to solid-state detecting unit 26 to move solid-state detecting unit 26 relative to opening 22, or relative to the prostate region of the patient, during data acquisition.

Processor 13, as depicted, may also comprise a controller 34 for controlling motion actuator 32.

Detector assembly 12 may include a structural imaging system. In the embodiment depicted in FIG. 19, the structural imaging system is an ultrasound system 42. As shown in FIG. 4, an ultrasound transducer 44 may be integrated into support surface 16 of chair 11 proximate opening 22, and/or ultrasound transducer 44 may be housed with CZT radiation detectors 30. In the bed configuration, ultrasound transducer 44 may extend from detector assembly 12 (see FIG. 10),In other embodiments, rather than an ultrasound system, the structural imaging system may, alternatively, be an x-ray system (not shown).

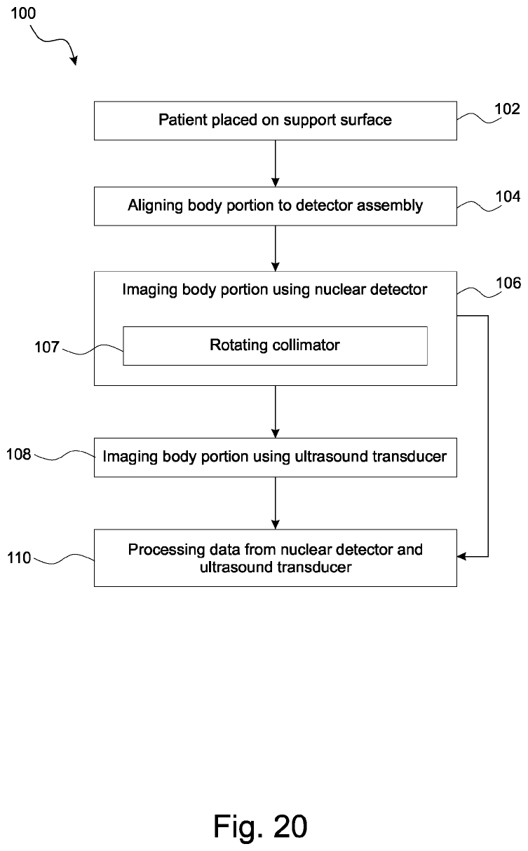

A method (100) to use imaging system 10/50 for non-invasive imaging of a body portion of a patient, is shown in FIG. 20. The patient is first placed on a surface for supporting the body portion of the patient that is to be imaged (102). With the present embodiments, the patient may be asked to sit in chair 11 or to lie on bed 52. The patient's body portion to be imaged, or in the present cases, the prostate region, is then aligned to be as close as possible to detector assembly 12 (104). In the case of the chair configuration, for example, the prostate region is aligned as close as possible to central opening 22. In the case of the bed configuration, the patient raises and separates his legs and shifts his prostate region or perineum towards detector assembly 12 on arm 54. Alternatively, arm 54 may be repositionally mounted to bed 52 such that detector assembly 12 may be brought towards patient 60 during alignment. Detector assembly 12 at all times remains external to the patient.

A medical radioisotope, such as Technetium-99m (^{99m}Tc) in combination with prostate-specific membrane antigen (PSMA) will have been injected into the patient. As the PSMA binds with the cancer cells in the prostate, the decaying Technetium-99m gives off gamma rays. As the gamma rays travel past the patient's perineum, they will be collimated by collimator 28 and certain rays will be detected by CZT radiation detectors 30, producing “hotspot” data (106). Collimator 28 limits the detected gamma rays to a pre-determined solid angle.

While Technetium-99m is a popular choice, other approved medical radioisotopes may be used instead.

Controller 34 and motion actuator 32 may be used to control the rotation of collimator 28 relative to CZT radiation detector 30 to change the solid angle during data acquisition. As collimator 28 and/or CZT radiation detector 30 is/are rotated (107) to pre-determined angles relative to one another, multiple tomographic images can be collected. Alternatively or additionally, motion actuator 32 may move solid-state detecting units 26 relative to the prostate region as data is acquired.

Ultrasound transducer 44 may also collect ultrasound data during the imaging step in order to collect structural data on the contours of the prostate and location within the prostate of a potential tumour (108). In some examples, imaging step includes two sub-steps: collecting data with CZT radiation detectors 30 and du collecting data with ultrasound transducer 44. These two imaging steps may be performed sequentially or simultaneously. CZT radiation detectors 30 and ultrasound transducer 44 may be mounted to respective support structures that can be independently moved to be proximate the body portion of the patient depending on which device is being used to collect data. CZT radiation detectors 30 and ultrasound transducer 44 may also be housed together in detector assembly 12. In example embodiments, ultrasound gel may be applied to the patient's body in the region that is to be imaged to facilitate ultrasound readings, i.e. the prostate region.

After processor 13 receives data from the one or more CZT radiation detectors 30 and ultrasound transducer 44, the data may be stored in memory for future processing. Processor 13 then uses a reconstruction overlay algorithm to process and fuse the data to produce a three-dimensional image that may show the contours of tumours and their three-dimensional position within the prostate (step 110).

In particular, two-dimensional (2D) images from the gamma detector, which identify hotspots, may be superpositioned onto a three-dimensional (3D) image from the ultrasound. The overlay software is typically based on the knowledge of the relative position of ultrasound transducer 44 and gamma radiation detector(s) 30. The relative positions are typically based on the physical specifications of the hardware of the system. This information allows the coordinate system of the ultrasound image and gamma image to be the same, making the overlay possible. In this manner, the projected column allows a user to identify tumour hotspots and, importantly, the location of the tumour(s) within the prostate.

As noted above, multiple tomographic images may be acquired during imaging. When multiple gamma detector are used and positioned relative to the body portion, or prostate, a 2D image may be reconstructed from each gamma detector during each scan. The more 2D images that are collected by the processor, the more information the user has to better identify a tumour's shape, size, and/or location.

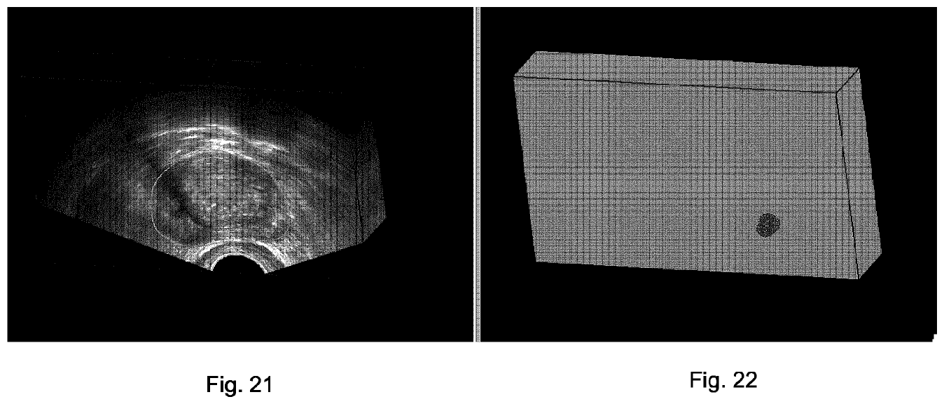

Such data tends to be valuable in aiding in the performance of biopsies. The fusion of data from CZT radiation detectors 30 and ultrasound transducer 44 can be particularly useful in some applications for providing a accurate 3-D representation of the scanned prostrate. FIGS. 21-23 are figures simulating the appearance of example images that may be produced using the above described systems, method, and reconstruction overlay process.

FIG. 21 shows a 3D ultrasound image of a prostate from the data collected by ultrasound transducer 44 and FIG. 22 shows a 3D image of radionuclide labels tracer registered to the particular 3D ultrasound image. The darker region of FIG. 22 shows a higher uptake of the tracer, i.e. a hotspot. Combining the two images via the reconstruction overlay software, the images can be overlaid over the same coordinate system (x, y, z).

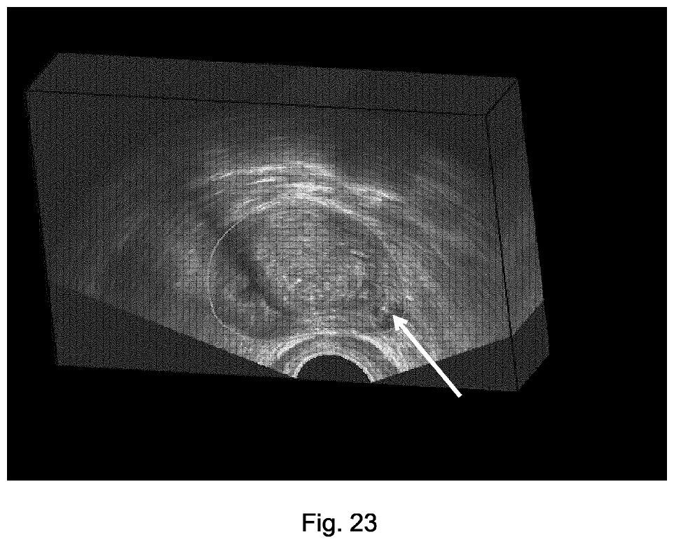

FIG. 23 shows the 3D ultrasound image of the prostate with the 3D image of the tracer superimposed. The tumour (indicated by the arrow) appears in the peripheral zone.

Once the reconstruction overlay has been completed and the reconstructed image array has been stored in the processor's memory, the image may be viewed and manipulated in a well known manner utilizing a volume viewer module.

An advantage of the present system is that the CZT radiation detectors 30 are positioned close to the patient's prostate, approximately 5 cm, to allow for effective gamma ray detection and, thus, may result in higher quality spatial resolution images. As well, given the price of CZT detectors, the present system may be more affordable than conventional nuclear imaging systems. Given that the system involves a simple chair or bed and relatively small detector assembly, the system tends to be easily handled and the method is simple to use. Another advantage is the fact that each of the detectors in the system does not require any trans-rectal imaging. The patient simply has to sit in the chair or lie on the bed, making the imaging process more comfortable for the patient.

In at least some examples, the imaging system could be adapted to examine other regions of the body, including posterior regions. For example, such posterior regions could include anatomical parts related to the urological and/or cervical systems of the body.

While some embodiments or aspects of the present disclosure may be implemented in fully functioning computers and computer systems, other embodiments or aspects may be capable of being distributed as a computing product in a variety of forms and may be capable of being applied regardless of the particular type of machine or computer readable media used to actually effect the distribution.

At least some aspects disclosed may be embodied, at least in part, in software. That is, some disclosed techniques and methods may be carried out in a computer system or other data processing system in response to its processor, such as a microprocessor, executing sequences of instructions contained in a memory, such as ROM, volatile RAM, non-volatile memory, cache or a remote storage device.

A computer readable storage medium may be used to store software and data which when executed by a data processing system causes the system to perform various methods or techniques of the present disclosure. The executable software and data may be stored in various places including for example ROM, volatile RAM, non-volatile memory and/or cache. Portions of this software and/or data may be stored in any one of these storage devices.

Examples of computer-readable storage media may include, but are not limited to, recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions can be embodied in digital and analog communication links for electrical, optical, acoustical or other forms of propagated signals, such as carrier waves, infrared signals, digital signals, and the like. The storage medium may be the internet cloud, or a computer readable storage medium such as a disc.

Furthermore, at least some of the methods described herein may be capable of being distributed in a computer program product comprising a computer readable medium that bears computer usable instructions for execution by one or more processors, to perform aspects of the methods described. The medium may be provided in various forms such as, but not limited to, one or more diskettes, compact disks, tapes, chips, USB keys, external hard drives, wire-line transmissions, satellite transmissions, internet transmissions or downloads, magnetic and electronic storage media, digital and analog signals, and the like. The computer usable instructions may also be in various forms, including compiled and non-compiled code.

At least some of the elements of the systems described herein may be implemented by software, or a combination of software and hardware. Elements of the system that are implemented via software may be written in a high-level procedural language such as object oriented programming or a scripting language. Accordingly, the program code may be written in C, C++, J++, or any other suitable programming language and may comprise modules or classes, as is known to those skilled in object oriented programming. At least some of the elements of the system that are implemented via software may be written in assembly language, machine language or firmware as needed. In either case, the program code can be stored on storage media or on a computer readable medium that is readable by a general or special purpose programmable computing device having a processor, an operating system and the associated hardware and software that is necessary to implement the functionality of at least one of the embodiments described herein. The program code, when read by the computing device, configures the computing device to operate in a new, specific and predefined manner in order to perform at least one of the methods described herein.

While the teachings described herein are in conjunction with various embodiments for illustrative purposes, it is not intended that the teachings be limited to such embodiments. On the contrary, the teachings described and illustrated herein encompass various alternatives, modifications, and equivalents, without departing from the described embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

1. An imaging system for tomographic imaging of a body portion of a patient, the system comprising:
a surface for supporting the body portion of the patient;
a detector assembly configured to be positioned proximate the body portion, the detector assembly comprising:
at least one solid state detecting unit comprising:
a nuclear detector; and
a collimator defining a solid collection angle; and
a processor for receiving collected data from the detector assembly.

2. The system of claim 1, wherein the detector assembly further comprises a structural imaging system to provide structural image data of the body portion to the processor.

3. (canceled)

4. The system of claim 2, wherein the processor is configured to process the collected data to generate nuclear image data and match coordinate systems of the structural image data and the nuclear image data and overlay the resulting images.

5. The system of claim 1 for non-invasive imaging of the prostate of the patient.

6. The system of claim 1, wherein the collimator is generally cylindrical and has multiple apertures arranged in a line which define the solid collection angle.

7. (canceled)

8. The system of claim 6, wherein each of the multiple apertures extends diametrically through the collimator.

9. The system of claim 6, wherein the detector assembly further comprises a motion actuator coupled to the at least one solid state detecting unit to rotate at least one of the collimator and the nuclear detector about an axis of the collimator during data acquisition.

10. (canceled)

11. The system of claim 1, wherein the nuclear detector is a cadmium zinc telluride (CZT) based radiation detector.

12. The system of claim 1, wherein the surface is on a seat, the seat:
having a support surface and an opposed surface, and
defining an opening; and
the detector assembly positioned proximate the opposed surface adjacent the periphery of the opening, wherein the detector assembly comprises a plurality of the solid state detection units positioned along the periphery proximate the opening.

13. (canceled)

14. The system of claim 1, wherein the surface is a horizontal surface, the detector assembly secured at an vertically extending angle to the horizontal surface of the bed.

15. A method for non-invasive imaging of a body portion of a patient, the method comprising:
aligning the body portion of the patient adjacent a detector assembly;
imaging the body region of the patient using at least one solid-state detecting unit within the detector assembly, the solid-state detecting unit comprising:
a nuclear detector; and
a collimator defining a solid collection angle; and
processing the collected image data from the detector assembly.

16. The method of claim 15, further comprising imaging the prostate region using an ultrasound transducer situated within the detector assembly.

17. The method of claim 16, wherein processing the collected image data comprises determining and matching coordinate systems of ultrasound image data and nuclear image data and overlaying the resulting images.

18. The method of claim 15, wherein the body portion is a prostate region of the patient.

19. The method of claim 15, further comprises rotating at least one of the collimator and the nuclear detector about an axis of the collimator during imaging.

20. (canceled)

21. An imaging system for tomographic imaging of a body portion of a patient, the system comprising:
a detector assembly configured to be positioned proximate the body portion, the detector assembly comprising:
a structural imaging system; and
at least one solid state detecting unit; and
a processor for receiving collected data from the detector assembly.

22. The system of claim 19, wherein the structural imaging system is an x-ray system or an ultrasound system; and the solid state detecting unit is a gamma detecting unit.

23. (canceled)

24. The system of claim 19, wherein the processor is configured to process the collected data to determine and match coordinate systems of ultrasound image data and nuclear image data and overlay the resulting images.

25. (canceled)

26. (canceled)

27. (canceled)

28. The system of claim 19, the solid state detecting unit comprising:
a collimator defining a solid collection angle; and
a nuclear detector at least partially surrounding the collimator.

29-34. (canceled)
